(19) United States
(12) Patent Application Publication
(10) Pub. No.: US 2025/0042838 A1
(43) Pub. Date: Feb. 6, 2025

(54) PROCESS FOR THE SYNTHESIS AND PURIFICATION OF CANNABINOIC ACIDS AND ACYLATED DERIVATIVES THEREOF

(71) Applicant: TRESCO LABS GMBH, Frankfurt am Main (DE)

(72) Inventor: Eric S. Cosky, Frankfurt am Main (DE)

(21) Appl. No.: 18/714,522

(22) PCT Filed: Nov. 30, 2022
(86) PCT No.: PCT/EP2022/083822

§ 371 Date: May 29, 2024

(30) Foreign Application Priority Data
Nov. 30, 2021 (EP) 21211470.6

(51) Int. Cl. C07C 69/78; A61K 31/00; C07C 67/00; C07C 67/52; C07C 69/88; C07D 311/80; C07D 493/04; C12P 7/22

(52) U.S. Cl. CPC C07C 69/78; A61K 31/658; C07C 67/00; C07C 67/52; C07C 69/88; C07D 311/80; C07D 493/04; C12P 7/22; C07C 2601/12; C07C 2601/16

(57) ABSTRACT

The invention relates to a novel process for the synthesis and/or purification of tetrahydrocannabinolic acid (THCA) alkanoates and/or cannabidiolic acid (CBDA) di-alkanoates. The invention also provides novel derivatives of CBDA di-alkanoates and THCA alkanoates according to Formula I, wherein R¹=methyl-4-(prop-1-en-2-yl)-cyclohex-1-ene-3-yl (limonenyl); wherein R²=acetate, propionate, butyrate, or OH; wherein R³=propyl, pentyl, or heptyl; wherein R⁴=methyl, ethyl, or propyl; wherein R⁵=methyl, ethyl, or propyl, or wherein R¹=methyl-4-(2-propyl)cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl, or wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

The invention relates to a novel process for the synthesis and/or purification of tetrahydrocannabinolic acid (THCA) alkanoates and/or cannabidiolic acid (CBDA) di-alkanoates. The invention also provides novel derivatives of CBDA di-alkanoates and THCA alkanoates.

### BACKGROUND

Cannabinoids are terpene phenols found in flowering Cannabinaceae plants of the genus Cannabis, namely in the species Cannabis sativa, Cannabis indica, and Cannabis ruderalis and are subdivided into 12 classes: cannabigerols (CBG), cannabichromenes (CBC), cannabidiols (CBD), cannabinodiols (CBND), tetrahydrocannabinols (THC), cannabinols (CBN), cannabitriols (CBT), cannabielsoins (CBE), isocannabinoids, cannabicyclols (CBL), cannabicitrans (CBT) and cannabichromanones (CBCN).

Cannabis extracts and concentrates (oils and resins) containing different amounts of different classes of cannabinoids depending on the extraction process have found wide applications as food additives and cosmetics. Some cannabinoids like, for instance, (−)-trans-cannabidiol (CBD) and (−)-Δ⁹-trans-tetrahydrocannabinol (Δ⁹-THC, Dronabinol) show distinct physiological effects by binding to the cannabinoid receptors CB1 and CB2 of the endocannabinoid system, and are also used as pharmaceuticals modulating appetite, pain, mood and memory (CB1) or influencing the immune system (CB2). The biosynthesis of the latter cannabinoids comprises the formation of cannabidiolic acid (CBDA) which is enzymatically decarboxylated to CBD or cyclized to tetrahydrocannabinolic acid (THCA). Decarboxylation of CBDA and THCA to CBD and THC, respectively also occurs chemically at elevated temperatures during the extraction process.

Methods for the isolation of cannabinoids are known in the art.

Formato et al. (Molecules 25, 2020, 2638) reviewed aspects on (−)-cannabidiolic acid. CBDA is bioactive and acts as an anti-inflammatory, anti-emetic, anti-convulsant and anti-carcinogenic drug. Formato et al. describe a procedure for extracting CBDA together with THCA from the “pollen”, or trichomes, of industrial hemp cultivars and managed to identify analytical quantities of purified CBDA using preparative thin-layer chromatography by which they distinguished CBDA from THCA. However, a solitary spot on a glass slide sprayed with silica, is not a convenient vehicle for arriving at even research quantities of an organic compound.

Current synthetic routes to CBDA, including CBDA purification from Hemp biomass, are still cumbersome and do not provide the desired purities.

Current methodologies to isolate and purify cannabinoids suffer to a large extent from the known fact that, with the exception of CBD, pure cannabinoids are typically oily compounds that are difficult, if not impossible, to crystallize. Additionally, valuable intermediates during synthesis and available through bio-production, such as THCA and CBDA, tend to decarboxylate to CBD and THC, respectively, during exposure to the elevated temperatures needed for extraction, vacuum distillation and even during prolonged storage at room temperature.

Accordingly, there is a need to further improve the existing technologies in terms of process simplicity, in order to guarantee compound integrity and obtain higher compound purity.

Furthermore, there is a related need to generate and isolate valuable intermediate compounds and provide novel cannabinoid derivatives that are accessible through the sought-after isolation and purification techniques.

The above objects are solved by the subject-matter of the claims and by the subject-matter described here below.

### SUMMARY OF THE INVENTION

In one aspect, the invention provides a compound according to Formula I

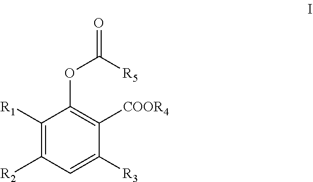

wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

The invention thus provides synthetic access to hitherto unknown cannabinoic acid derivatives which have been further converted into novel THC derivatives.

In a further aspect, the invention provides a process for the synthesis and/or purification of tetrahydrocannabinolic acid (THCA) alkanoate and/or cannabidiolic acid (CBDA) di-alkanoate, preferably tetrahydrocannabinolic acid (THCA) acetate and/or cannabidiolic acid (CBDA) di-acetate, comprising the steps of
providing a crude plant extract containing a mixture of cannabinoic acids, or providing CBDA;
contacting and/or reacting the crude plant extract or the CBDA with an acylation agent, preferably an acetylation agent, and a chemical base, to obtain THCA alkanoate and/or CBDA di-alkanoate, preferably THCA acetate and/or CBDA di-acetate; and
isolating the obtained THCA alkanoate and/or CBDA di-alkanoate, preferably THCA acetate and/or CBDA di-acetate,
wherein the acylation agent is selected from the list of C₁-C₁₀ aliphatic or aromatic anhydrides, preferably acetic anhydride,
wherein the chemical base is preferably a tertiary amine, such as pyridine or preferably triethylamine,
wherein isolation is carried out by means of crystallization.

This chemical method for the isolation of CBDA from crude plant extracts via the preparation of derivatives of CBDA and THCA is simple, efficient and also allows separating CBDA from a diverse mixture of chemical carboxylation products of CBD.

The method according to the invention circumvents time consuming and laborious chromatographic separations of cannabinoic acids by isolating the sought-after compounds through simple crystallization of chemically modified derivatives.

The method according to the invention is advantageous because it provides THCA acetate and CBDA di-acetate in pure and crystalline form which is and makes them distinct over many other cannabinoids which are oily and escape crystallization due to their chemical nature. Additionally, the method circumvents and/or solves the problem that THCA is labile and very prone to decarboxylation. Specifically, THCA acetate cannot decarboxylate and is much more stable than THCA.

In a further aspect, the invention provides a compound according to Formula I for use in medicine.

Cannabinoic acid derivatives have shown wide-ranging health benefits and applications in the medical field, and may serve to treat people and/or animals suffering from inflammation, nausea, convulsion, or even cancer.

### DETAILED DESCRIPTION

### Novel Derivatives of CBDA and THCA

In one aspect, the invention provides a compound according to Formula I

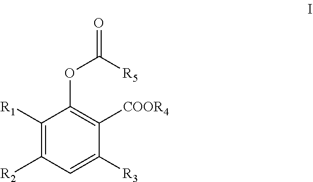

wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

The invention thus provides synthetic access to hitherto unknown cannabinoic acid derivatives which have been further converted into novel THC derivatives.

The provided compounds derive their utility from the fact that they are both crystalline and their decarboxylation is hindered through esterification of the phenolic alcohol adjacent to the carboxylic acid moiety and esterification of the carboxylic acid itself, which increases both their processability and stability. In contrast, the natural products with the acid functionality are labile and decompose at temperatures above 50° C. The combined properties make them highly suitable with regard to cannabinoid extraction, purification and/or isolation.

In one embodiment, a compound is provided having a Formula I

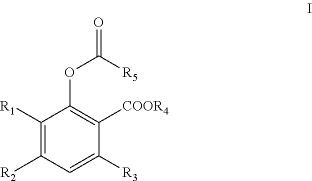

wherein R¹=methyl-4-(prop-1-en-2-yl)-cyclohex-1-ene-3-yl (limonenyl);
wherein R²=acetate, or OH;
wherein R³=propyl, pentyl, or heptyl;
wherein R⁴=methyl;
wherein R⁵=methyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, and wherein R⁵=methyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

The identified compounds are crystalline and thereby enable the purification and isolation of e.g. CBDA-di-acetate and further homologues according to Formula I from crude plant extracts. These compounds are value-added intermediates conducive to exceptionally high-yielding cyclisation and purity for the purpose of creating further derivatives, such as e.g. THCA, Tetrahydrocannabivarinic acid (THCVA), delta(9)-Tetrahydrocannabiphorolic acid (THCPA), THC, Tetrahydrocannabivarin (THCV), and Tetrahydrocannabiphorol (THCP).

In one embodiment, a compound is provided having Formula II

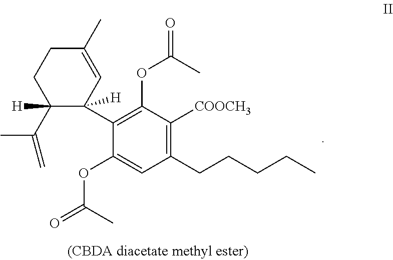 [SMILES: C=C(C)[C@@H]1CCC(C)=C[C@H]1c1c(OC(C)=O)cc(CCCCC)c(C(=O)OC)c1OC(C)=O]

In one embodiment, a compound is provided having Formula III

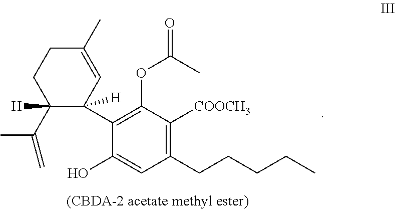 [SMILES: C=C(C)[C@@H]1CCC(C)=C[C@H]1c1c(O)cc(CCCCC)c(C(=O)OC)c1OC(C)=O]

The compound according to Formula III is valuable as it allows subsequent derivatization into a THCA derivative, i.e. compound according to Formula IVa.

In one embodiment, a compound is provided having Formula IVa

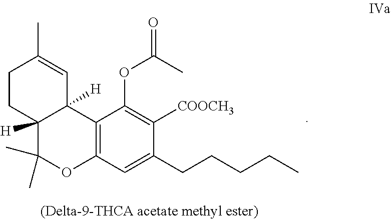 [SMILES: CCCCCc1cc2c(c(OC(C)=O)c1C(=O)OC)[C@@H]1C=C(C)CC[C@H]1C(C)(C)O2]

In one embodiment, a compound is provided having Formula IVb

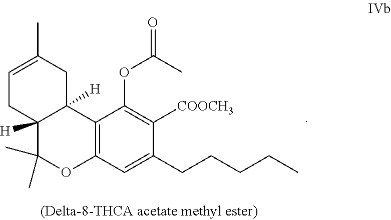 [SMILES: CCCCCc1cc2c(c(OC(C)=O)c1C(=O)OC)[C@@H]1CC(C)=CC[C@H]1C(C)(C)O2]

In one embodiment, a compound is provided having Formula V

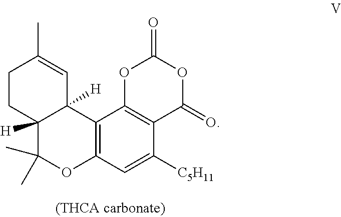

The compound according to Formula V is highly valuable as it is a non-psychoactive derivative, which is further amenable to hydrolysis upon which it decomposes into THCA and CO₂. Hydrolysis of the compound according to Formula V can be effected by exposure to water or atmospheric moisture.

### Pharmaceutical Composition

In a further aspect, the invention provides a pharmaceutical composition comprising a compound according to Formula I

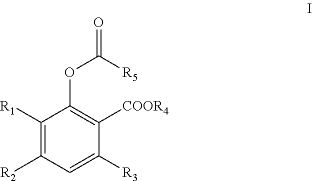

wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other, in a pharmaceutically acceptable carrier.

In one embodiment, a pharmaceutical composition is provided comprising a compound selected from the list of

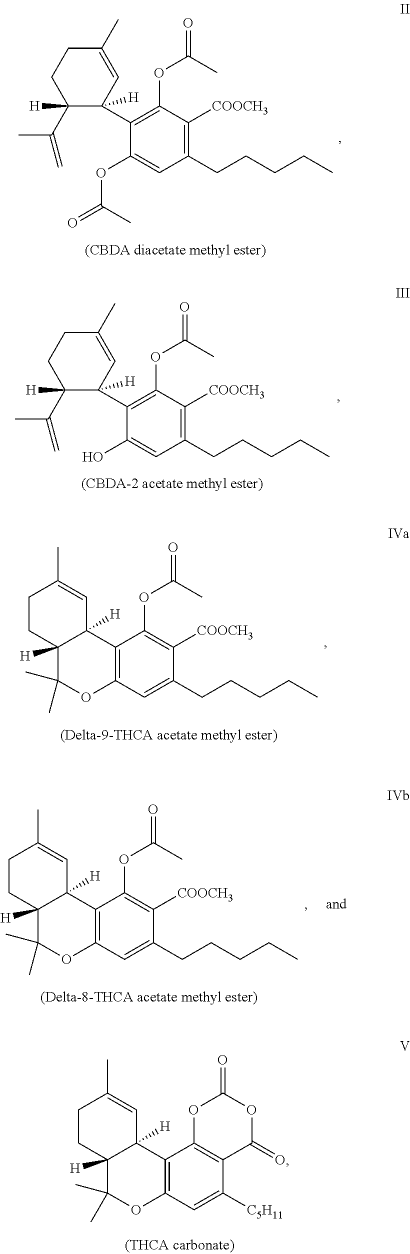

in a pharmaceutically acceptable carrier.

### Process

In a further aspect, the invention provides a process for the synthesis and/or purification of tetrahydrocannabinolic acid (THCA) alkanoate and/or cannabidiolic acid (CBDA) di-alkanoate, preferably tetrahydrocannabinolic acid (THCA) acetate and/or cannabidiolic acid (CBDA) di-acetate, comprising the steps of
providing a crude plant extract containing a mixture of cannabinoic acids, or providing CBDA;
contacting and/or reacting the crude plant extract or the CBDA with an acylation agent, preferably an acetylation agent, and a chemical base, to obtain THCA alkanoate and/or CBDA di-alkanoate, preferably THCA acetate and/or CBDA di-acetate; and
isolating the obtained THCA alkanoate and/or CBDA di-alkanoate, preferably THCA acetate and/or CBDA di-acetate,
wherein the acylation agent is selected from the list of C₁-C₁₀ aliphatic or aromatic anhydrides, preferably acetic anhydride,
wherein the chemical base is preferably a tertiary amine, such as pyridine or preferably triethylamine,
wherein isolation is carried out by means of crystallization.

This chemical method for the synthesis and/or purification of tetrahydrocannabinolic acid (THCA) alkanoate and/or cannabidiolic acid (CBDA) di-alkanoate allows the isolation of THCA and/or CBDA from crude plant extracts via the preparation of derivatives of CBDA and/or THCA. The process according to the invention is simple, efficient and also allows separating THCA and/or CBDA from a diverse mixture of chemical carboxylation products of for example CBD. Specifically, the process circumvents time consuming and laborious chromatographic separations of cannabinoic acids by isolating the sought-after compounds through crystallization of chemically modified derivatives.

The method according to the invention is advantageous because it provides THCA alkanoate and/or CBDA di-alkanoate, preferably THCA acetate and/or CBDA di-acetate, in pure and crystalline form which makes these compounds and their derivatives distinct over many other cannabinoids which are oily and escape crystallization due to their chemical nature. These acid-form alkanoates are protected from decarboxylation, and offer greater thermal stability for their manipulation and storage. In this respect, the method circumvents and/or solves the problem that THCA is labile and very prone to decarboxylation. Specifically, THCA acetate cannot decarboxylate and is much more stable than THCA.

If this disclosure refers to a step of “contacting and/or reacting” a crude plant extract or “reacting” or “treating” any other starting compound, including any intermediate compound, with a chemical agent or a number of chemical agents, optionally in a suitable solvent, it shall be understood that each step or sequence of steps is carried out for a time sufficient to effect (i.e. allow) the underlying reaction (to take place) of at least a portion of the starting compound(s) into the target (or intermediate) compound. Preferably, the time is sufficient to allow the underlying reaction to take place and to reach chemical equilibrium between the starting compound(s) and the target (or intermediate) compounds at the respectively chosen reaction or treatment conditions. Analogously, the skilled person understands and knows how to carry out the addition of chemical (re)agents under e.g. stirring and/or maintaining a preferred temperature in order to optimise and/or maximise the yields of the target compounds. Furthermore, the skilled person understands and knows how to carry out the work-up of reaction batches, e.g. adding water and/or combining with an aqueous HCl solution for neutralisation purposes, extraction using organic solvents, washing steps with e.g. aqueous HCl solution or with saturated NaCl solution, drying over Na₂SO₄, filtration and concentration, in order to optimise and/or maximise the yields of the target compounds.

In one embodiment, the crude plant extract is provided by enzymatic extraction. Advantageously, during an enzymatic extraction, not as much heat is generated so that decarboxylation-sensitive material is not decarboxylated.

In one embodiment, providing a crude plant extract containing a mixture of cannabinoic acids comprises providing hemp pollen, preferably by isolating the trichomes from the plant inflorescences optionally by a sieving process, and by subjecting the hemp pollen to an enzymatic process, wherein the cellulose of the plant cell walls is hydrolyzed and/or decomposed enzymatically to release cannabinoids into an aqueous solution, at a temperature between 1° and 40° C., preferably between 2° and 30° C. Advantageously, the enzymatic process preserves CBDA in its acid form and prevents it from decarboxylation.

In one embodiment, providing a crude plant extract containing a mixture of cannabinoic acids comprises providing hemp pollen, preferably by isolating the trichomes from the plant inflorescences optionally by a sieving process, by subjecting the hemp pollen to ultrasound accelerated maceration using alternating pressure and cavitation to effect at least partial cell decomposition and/or at least partial release of intracellular metabolic content, and by extracting the cannabinoic acids using a solvent, preferably a chloroform/n-hexane (1:1, v/v) solvent mixture, at a temperature between 1° and 40° C., preferably between 2° and 30° C. Advantageously, the cold extraction process preserves CBDA in its acid form and prevents it from decarboxylation.

In one embodiment, the step extracting the cannabinoic acids using a solvent, preferably a chloroform/n-hexane (1:1, v/v) solvent mixture, is performed batch-wise and using per batch between 10 g to 50 g hemp pollen (HP) with an HP to solvent (or solvent mixture) ratio of between 1:4 and 1:6 (w/w) and employing three extraction cycles. Preferably each extraction cycle lasts at least 10 min, more preferably at least 30 min.

In one embodiment, contacting and/or reacting the crude plant extract with an acylation agent, preferably an acetylation agent, and a chemical base, wherein the acylation agent is selected from the list of C₁-C₁₀ aliphatic or aromatic anhydrides, preferably acetic anhydride, and wherein the chemical base is preferably a tertiary amine, such as pyridine or preferably tri-ethylamine, comprises dissolving the crude plant extract by shaking at room temperature in a suitable solvent, such as e.g. DMF, adding the acylation agent dropwise under stirring and maintaining the solvent temperature below 50° C., and allowing further contact and/or reaction for 1 to 4 hours, preferably 1.5 to 2.5 hours, at room temperature. This sequence of method steps provides for a synthesis to obtain THCA alkanoate and/or CBDA di-alkanoate, preferably THCA acetate and/or CBDA di-acetate.

If this disclosure refers to room temperature, a temperature range between 18 and 30° C., preferably between 21 and 24° C., shall be understood and employed.

In one embodiment, the acylation agent is selected from the list of C₁-C₁₀ aliphatic or aromatic anhydrides, such as e.g. propionic anhydride, butyric anhydride, pentanoic anhydride, hexanoic anhydride, heptanoic anhydride, octanoic anhydride, nonaoic anhydride, decanoic anhydride, benzoyl anhydride and substituted benzoyl anhydride, preferably acetic anhydride.

In one embodiment, the chemical base is a tertiary amine, such as e.g. pyridine, diisopropylethylamine or preferably triethylamine. Advantageously, triethylamine is inexpensive, easy to remove from the reaction mixture, and has further benefits over many chemical bases that may be used for the process, such as e.g. pyridine.

In one embodiment of the process, a further step may be included after the step contacting and/or reacting the crude plant extract with an acylation agent and a chemical base, comprising cooling to 0° C., adding water to the contacted and/or reacted crude plant extract, e.g. the reaction mixture, under stirring for 0.5 h at room temperature, combining with an aqueous HCl solution (around 2 M), extracting twice with ethyl acetate, washing the combined ethyl acetate extracts with aqueous HCl solution (around 2 M), followed by washing three times with saturated NaCl solution, drying over Na₂SO₄, filtration and concentration.

In one embodiment of the process, isolating the obtained THCA alkanoate and/or CBDA di-alkanoate, preferably THCA acetate and/or CBDA di-acetate, is carried out by means of crystallization. The skilled artisan knows and understands that crystallization encompasses complete dissolution of a crude product or material in a solvent, preferably by heating the solvent to effect and/or accelerate dissolution but establishing and/or maintaining a temperature below the decomposition temperature of the crude product or material, optional filtration through e.g. a layer of silica gel, and subsequent cooling to obtain crystals of the product or material.

Advantageously, THCA alkanoates and/or CBDA di-alkanoates, preferably THCA acetate and/or CBDA di-acetate, are amenable to crystallisation, whereas most other cannabinoid compounds and derivatives are oily, difficult to work with and difficult to process. In particular, the crystallisation of THCA alkanoates and/or CBDA di-alkanoates, preferably THCA acetate and/or CBDA di-acetate, is feasible and simple.

With respect to CBDA and THCA, the free non-derivatised acids cannot be crystallised, because heating would lead to decarboxylation. Advantageously, THCA alkanoates and/or CBDA di-alkanoates, preferably THCA acetate and/or CBDA di-acetate, can be crystallised without decarboxylation, and to thus leave intact the chemical derivative.

In one embodiment of the process, crystallization is performed in n-pentane or n-hexane. Advantageously, the low boiling points of n-pentane or n-hexane, in particular n-pentane, provide for and support crystallisation conditions that leave the derived compounds, i.e. THCA alkanoates and/or CBDA di-alkanoates, preferably THCA acetate and/or CBDA di-acetate, intact, and may thereby inherently prevent decay reactions, such as decarboxylation.

In one embodiment, a process for the synthesis and/or purification of tetrahydrocannabinolic acid (THCA) alkanoate, preferably tetrahydrocannabinolic acid (THCA) acetate, is provided comprising the steps of
providing a crude plant extract containing a mixture of cannabinoic acids;
contacting and/or reacting the crude plant extract with an acylation agent, preferably an acetylation agent, and a chemical base, to obtain THCA alkanoate, preferably THCA acetate; and
isolating the obtained THCA alkanoate, preferably THCA acetate,
wherein the acylation agent is selected from the list of C₁-C₁₀ aliphatic or aromatic anhydrides, preferably acetic anhydride,
wherein the chemical base is preferably a tertiary amine,
wherein isolation is carried out by means of crystallization.

In one embodiment, a process for the synthesis and/or purification of cannabidiolic acid (CBDA) di-alkanoate, preferably cannabidiolic acid (CBDA) di-acetate, is provided comprising the steps of
providing CBDA;
contacting and/or reacting the CBDA with an acylation agent, preferably an acetylation agent, and a chemical base, to obtain CBDA di-alkanoate, preferably CBDA di-acetate;
and
isolating the obtained CBDA di-alkanoate, preferably CBDA di-acetate,
wherein the acylation agent is selected from the list of C₁-C₁₀ aliphatic or aromatic anhydrides, preferably acetic anhydride,
wherein the chemical base is preferably a tertiary amine,
wherein isolation is carried out by means of crystallization.

In one embodiment, the process comprises the further step of treating the obtained CBDA diacetate with an alkylation agent, such as e.g. C₁-C₁₀ dialkylsulfate, preferably dimethyl sulfate, to obtain a first CBDA di-acetate derivative, preferably CBDA diacetate methyl ester according to Formula II

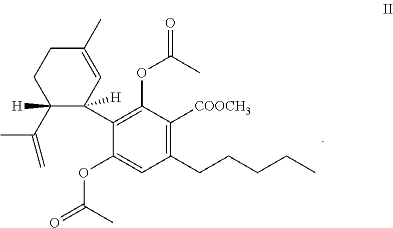 [SMILES: C=C(C)[C@@H]1CCC(C)=C[C@H]1c1c(OC(C)=O)cc(CCCCC)c(C(=O)OC)c1OC(C)=O]

In one embodiment, the step of treating the obtained CBDA di-acetate with an alkylation agent comprises dissolving CBDA-di-acetate in a solvent, preferably acetonitrile, and adding an excess of triethylamine, preferably 1.1 mol equivalents with respect to CBDA-di-acetate, while stirring the solution at room temperature, drop-wise adding an excess of dimethyl sulfate, preferably 1.1 mol equivalents with respect to CBDA-di-acetate, into the stirred solution, maintaining a temperature at or below 50° C., further stirring the solution at room temperature.

In an alternative embodiment, the step of treating the obtained CBDA di-acetate with an alkylation agent comprises dissolving CBDA-di-acetate in a solvent, preferably acetonitrile, and adding an excess of triethylamine, preferably 1.1 mol equivalents with respect to CBDA-di-acetate, while stirring the solution at room temperature, drop-wise adding an excess of dimethyl sulfate, preferably 1.1 mol equivalents with respect to CBDA-di-acetate, into the stirred solution, maintaining a temperature at around 60° C. for 2 h, and pouring the reaction solution into ice-cold 1 M aqueous HCl solution, and extracting the resulting suspension twice with dichloromethane, washing the combined organic extracts with 1 M aqueous HCl solution, saturated aqueous NaHCO₃ solution and saturated aqueous NaCL solution and drying over Na₂SO₄.

In one embodiment, the process comprises the further step of deacylating the first CBDA di-acetate derivative to obtain a CBDA mono-acetate derivative, preferably CBDA-2-acetate methyl ester according to Formula III

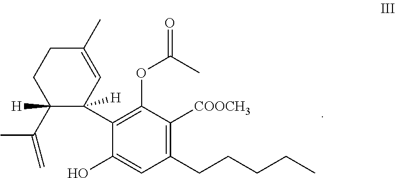 [SMILES: C=C(C)[C@@H]1CCC(C)=C[C@H]1c1c(O)cc(CCCCC)c(C(=O)OC)c1OC(C)=O]

In one embodiment, the step of deacylating the first CBDA di-acetate derivative comprises dissolving the first CBDA di-acetate derivative in an alcohol, e.g. methanol, adding a base reagent, e.g. sodium methanolate or a solution of ammonia in methanol, and stirring the solution at room temperature for 24 h. In one embodiment relying on sodium methanolate, the process comprises the further steps of neutralizing the solution by addition of Amberlyst IR 120 ion exchange resin (in its protonated form), filtration and concentration. In one embodiment relying on ammonia, the process comprises the further steps of concentrating the solution.

In one embodiment, the process comprises the further step of converting the CBDA mono-acetate derivative into a THCA mono-acetate derivative, preferably THCA 2-acetate methyl ester according to Formula IVa

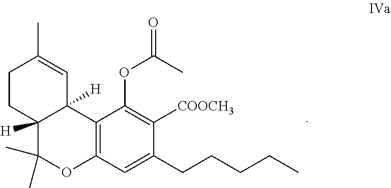 [SMILES: CCCCCc1cc2c(c(OC(C)=O)c1C(=O)OC)[C@@H]1C=C(C)CC[C@H]1C(C)(C)O2]

In one embodiment, the step of converting the CBDA mono-acetate derivative into a THCA mono-acetate derivative comprises dissolving the CBDA mono-acetate derivative in dichloromethane and adding boron trifluoride diethyl etherate, stirring under a protection gas atmosphere, preferably a nitrogen atmosphere, and washing with saturated aqueous NaHCO₃ solution, 2 N aqueous HCl solution, saturated aqueous NaCl solution, drying over Na₂SO₄ and filtration. In a further embodiment, the process comprises the additional steps of concentrating the filtrate and recrystallization from n-hexane.

In one embodiment, the step of converting the CBDA mono-acetate derivative into a THCA mono-acetate derivative comprises dissolving the CBDA mono-acetate derivative in dichloromethane and adding boron trifluoride diethyl etherate together with a mixture of trialkyl amine, preferably diisopropyl ethylamine, and tetraethylene glycol dimethyl ether, stirring under a protection gas atmosphere, and washing with saturated aqueous NaHCO₃ solution, 2 N aqueous HCl solution, saturated aqueous NaCl solution, drying over Na₂SO₄ and filtration. In a further embodiment, the process comprises the additional steps of concentrating the filtrate and recrystallization from n-hexane.

In one embodiment, a process is disclosed comprising the step of converting CBD mono-acetate into THC mono-acetate, wherein the process comprises dissolving the CBD mono-acetate in dichloromethane and adding boron trifluoride diethyl etherate together with a mixture of trialkyl amine, preferably diisopropyl ethylamine, and tetraethylene glycol dimethyl ether, stirring under a protection gas atmosphere, and washing with saturated aqueous NaHCO₃ solution, 2 N aqueous HCl solution, saturated aqueous NaCl solution, drying over Na₂SO₄ and filtration. In a further embodiment, the process comprises the additional steps of concentrating the filtrate and recrystallization from n-hexane.

The use of a mixture of trialkyl amine, preferably diisopropyl ethylamine, and tetraethylene glycol dimethyl ether, moderates the activity of the boron trifluoride diethyl etherate, and thereby enables the kinetically controlled production of delta-9 THC to predominate over delta-8 THC.

This approach hence creates an environment through which Lewis acid-catalyzed cyclization of CBD (or CBDA) leads to the kinetically controlled synthesis of delta-9 THC (or delta-9 THCA) from enantiomerically pure CBD or CBDA.

In contrast, under thermodynamic control, protonation of the cyclohexene double bond in CBD or CBDA facilitates tertiary carbenium ion formation which drives delta-8 THC production to the thermodynamic equilibrium existing between delta-8 THC and delta-9 THC which is at a ratio of 97/3.

In one embodiment, the mixture of trialkyl amine, preferably diisopropyl ethylamine, and tetraethylene glycol dimethyl ether comprises a volume/volume ratio of 30/70 to 70/30 diisopropyl ethylamine/tetraethylene glycol dimethyl ether, preferably a volume/volume ratio of 40/60 to 60/40 diisopropyl ethylamine/tetraethylene glycol dimethyl ether, more preferably a volume/volume ratio of 45/55 to 55/45 diisopropyl ethylamine/tetraethylene glycol dimethyl ether, and most preferably a volume/volume ratio of 50/50 diisopropyl ethylamine/tetraethylene glycol dimethyl ether.

In one embodiment, the process comprises the further step of converting THCA 2-acetate methyl ester, preferably via a THCA intermediate, into THCA carbonate according to Formula V

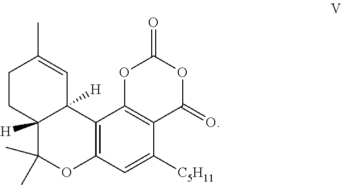

In one embodiment, the step of converting THCA 2-acetate methyl ester comprises dissolving and stirring THCA 2-acetate methyl ester in a solution of 0.5 to 2.0 M LiOH, preferably about 1 M LiOH, in an alcohol, preferably methanol, for 12 to 36 h, preferably about 24 h. In a further embodiment, the process comprises the additional steps of reacting with an aqueous HCl solution, preferably of 2 M concentration, extracting twice with dichloromethane, and washing the combined extracts with saturated aqueous NaCl solution, drying over Na₂SO₄, filtration and concentration to obtain crude THCA.

In a further embodiment, the process comprises the steps of dissolving the crude THCA in an aromatic solvent, such as e.g. toluene, adding a base reagent, e.g. trimethylamine and phosgene, preferably 2 M phosgene, stirred the solution at room temperature, washing with saturated aqueous NaHCO₃ solution, drying over Na₂SO₄, filtration and concentration, to obtain THCA carbonate.

In one embodiment of the process, the obtained derivatives are at least 70% pure, preferably 90% pure, and more preferably 99% pure, wherein the obtained derivatives are selected from

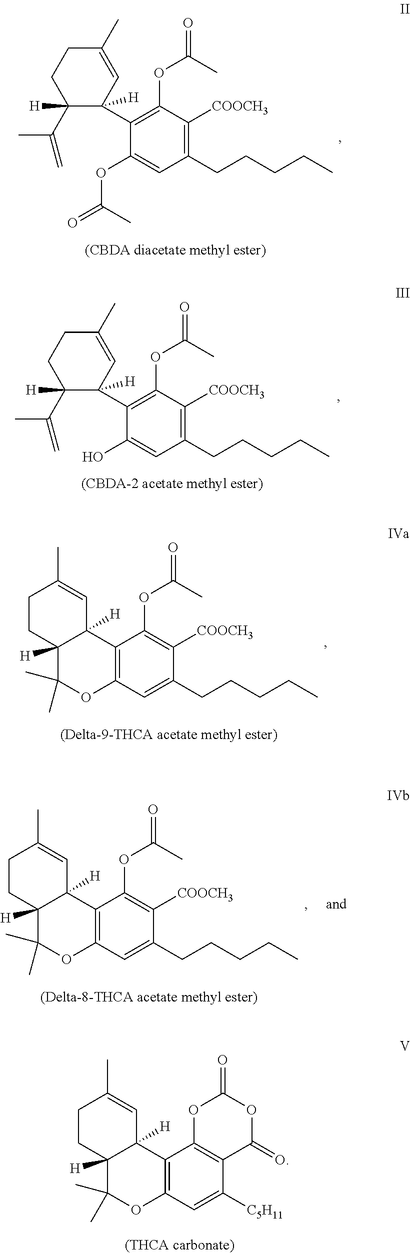

In one embodiment of the process, a distillation step is absent and/or a chromatography step is absent. Advantageously, chromatography steps are avoided because such measures are tedious and expensive. Advantageously, also a distillation step is absent because the compounds described and provided herein are heat-labile and may suffer from decomposition at increased temperatures, with reference to room temperature, preferably with reference to room temperature plus 20° C. In particular, the absence of a distillation step may prevent decarboxylation of THCA alkanoate and/or CBDA di-alkanoate, preferably THCA acetate and/or CBDA di-acetate, during or as part of the process.

In one embodiment of the process, the plant extract is obtained from a plant of the species Cannabinaceae, preferably a plant selected from the list of Cannabis sativa, Cannabis indica, and Cannabis ruderalis. Plant extracts obtained from the named plant species are naturally rich in cannabinoids and thus lend themselves to provide for the synthesis and/or purification of THCA alkanoate and/or CBDA di-alkanoate, preferably THCA acetate and/or CBDA di-acetate.

In another aspect, this disclosure provides a process for the conversion of CBD into Delta-9-THC comprising the steps of
providing a first solution comprising CBD in an organic solvent;
providing a second solution comprising tetraethylene glycol dimethyl ether, di-isopropyl ethylamine and boron trifluoride diethyl ether complex dissolved in the organic solvent;
adding the first solution to the second solution to obtain a reaction mixture;
optionally adding a further portion of the second solution to the reaction mixture; and
obtaining Delta-9-THC,
   wherein the process is carried out at a temperature between 1° and 40° C., preferably between 2° and 30° C., wherein the process is carried out under a protection gas atmosphere.

In one embodiment, the protection gas atmosphere is a nitrogen atmosphere. In an alternative embodiment, the protection gas atmosphere is an argon atmosphere.

In one embodiment, this disclosure provides a process for the conversion of CBD into Delta-9-THC comprising the steps of
providing a first solution comprising CBD in an organic solvent;
providing a second solution comprising tetraethylene glycol dimethyl ether, di-isopropyl ethylamine and boron trifluoride diethyl ether complex dissolved in the organic solvent;
adding the first solution to the second solution to obtain a reaction mixture;
optionally adding a further portion of the second solution to the reaction mixture;
stirring the reaction mixture for at least one hour, preferably at least three hours; and
obtaining Delta-9-THC,
   wherein the process is carried out at a temperature between 1° and 40° C., preferably between 2° and 30° C., wherein the process is carried out under a protection gas atmosphere.

In one embodiment, the process for the conversion of CBD into Delta-9-THC comprises the further steps of
washing the reaction mixture with a neutralizing solution, e.g., an aqueous hydrogen chloride solution;
repeated washing of the reaction mixture with a NaCl solution; and
drying the reaction mixture, for example over Na₂SO₄, optional filtration of the reaction mixture, and evaporation of the organic solvent.

In a further aspect, this disclosure provides a process for the synthesis of cannabidiolic acid (CBDA) comprising the steps of
providing a solution comprising CBD in an organic solvent;
adding a non-nucleophilic base to the solution, wherein the non-nucleophilic base is preferably DBU (1,8-Diazabicyclo[5.4.0]undec-7-ene); and
gassing carbon dioxide, preferably via bubbling, through the solution to obtain CBDA.

This approach provides CBDA at high purity and with good yields.

In one embodiment, this disclosure provides a process for the synthesis of cannabidiolic acid (CBDA) comprising the steps of
providing a solution comprising CBD in an organic solvent, such as e.g., acetonitrile and DMF;
adding a non-nucleophilic base to the solution, wherein the non-nucleophilic base is preferably DBU (1,8-Diazabicyclo[5.4.0]undec-7-ene); and
gassing carbon dioxide, preferably via bubbling, through the solution to obtain CBDA;
washing the solution with a neutralizing solution, e.g., an aqueous hydrogen chloride solution;
optionally repeated washing of the solution with a NaCl solution; and
extraction of the solution into a second organic solvent to obtain an extract, optionally drying the extract, and evaporation of the second organic solvent from the extract to obtain CBDA, preferably in crystalline form.

### Medical Use

In a further aspect, the invention provides a compound according to Formula I for use in medicine, wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

In a further aspect, the invention provides a compound according to Formula II for use in medicine.

In a further aspect, the invention provides a compound according to Formula III for use in medicine.

In a further aspect, the invention provides a compound according to Formula IVa for use in medicine.

In a further aspect, the invention provides a compound according to Formula IVb for use in medicine.

In a further aspect, the invention provides a compound according to Formula V for use in medicine.

Cannabinoic acid derivatives have shown wide-ranging health benefits and applications in the medical field, and may serve to treat people and/or animals suffering from inflammation, nausea, convulsion, or even cancer.

In one embodiment, a compound according to Formula I is provided for use in treating or preventing a disease associated with a cannabinoid receptor in a subject in need thereof, wherein the cannabinoid receptor is one or more of CB1, CB2, 5HT1A, 5HT2A, GPR18, GPR55, GPRI 19, TRPVI, TPRV2, PPARγ or a μ-opioid receptor.

In one embodiment, a compound according to Formula II, Formula III, Formula IVa, Formula IVb, or Formula V is provided for use in treating or preventing a disease associated with a cannabinoid receptor in a subject in need thereof, wherein the cannabinoid receptor is one or more of CB1, CB2, 5HT1A, 5HT2A, GPR18, GPR55, GPRI 19, TRPVI, TPRV2, PPARγ or a μ-opioid receptor.

In one embodiment, a compound according to Formula I is provided for use in treating or preventing a disease associated with a cannabinoid receptor in a subject in need thereof, wherein the cannabinoid receptor is CB1 and/or CB2.

In one embodiment, a compound according to Formula II, Formula III, Formula IVa, Formula IVb, or Formula V is provided for use in treating or pre-venting a disease associated with a cannabinoid receptor in a subject in need thereof, wherein the cannabinoid receptor is CB1 and/or CB2.

Cannabinoids, as well as their derivatives, are compounds active on cannabinoid receptors in humans and animals and have been implicated in many of the pharmacological benefits underlying the medical conditions, such as chronic pain, epilepsy, sleep disorders, anxiety, cancer, glaucoma, nausea, amyotrophic lateral sclerosis (ALS), Alzheimer's disease, Crohn's disease, Post-traumatic Stress Disorder (PTSD), arthritis, and fibromyalgia.

CB1 and CB2 receptors, as well as other receptors have been implicated in modulating the activity of cannabinoids in the human and/or animal body. Serotonin receptors, such as 5HT1A and 5HT2A, are known targets of cannabinoids.

Both receptor classes, i.e. CB1/CB2 receptors and the serotonin receptors, have been implicated with several neurological phenomena, such as anxiety, appetite, cognition, and mood.

Further receptors, such as e.g. GPR18, GPR55, GPR119, TRPV1, TPRV2, PPARs, and μ-opioid receptors are regulated and/or influenced to at least some extent by cannabinoid or cannabinoid-derived compounds.

In one embodiment, this disclosure provides a compound according to Formula I for use in the treatment of conditions relating to or benefiting from COX-2 inhibition, wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
   or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
   or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ vanish, i.e. are radicals forming a direct bond to each other.

In one embodiment, this disclosure provides CBDA-2-acetate for use in the treatment of conditions relating to or benefiting from COX-2 inhibition.

COX-2 inhibition counteracts inflammation and inflammatory processes.

### EXAMPLES

### Extraction of Crude Plant Material

The crude plant extract is prepared via an enzymatic polysaccharide hydrolyzing process (Herbolea) or a “cold” extraction process, both processes preserving CBDA from decarboxylation.

### Method 1

Dry hemp (25 g) was ground in a mortar and suspended in n-hexane (1 l). The mixture was stirred at room temperature for 3 h followed by sonification in an ultrasound bath for 15 min. The resulting slurry was filtered through a paper filter and the filtrate was concentrated in vacuo at a rotary evaporator (bath temperature 30° C.) to ca. 200 ml. The light red solution was extracted with 1% aqueous NaOH solution (150 ml) and the alkaline aqueous extract was poured into ice cold 10% HCl solution (200 ml). The resulting mixture was extracted with ethyl acetate (2×50 ml), the organic extracts washed with saturated NaCl solution, dried over Na₂SO₄, filtered and concentrated.

### Method 2

Hemp pollen (HP) is a resin produced by isolating the trichomes from the plant inflorescences by a sieving process and was used as a CBDA source. Hemp pollen, obtained from Hemp Farm Lab farmers (Caserta, Italy), was subjected to ultrasound accelerated maceration (Branson UItrasonics™ M3800-E, Danbury, CT, USA). Alternating pressure and cavitation was used for cell decomposition and the release of the intracellular metabolic content. A chloroform/n-hexane (1:1, v/v) solvent mixture was used as extracting solution, using per batch around 10 g HP with an HP to solvent mixture ratio of 1:5 and three extraction cycles. Each extraction cycle lasted 30 min. The obtained compound mixture was enriched with cannabinoids at a yield of at least 25%.

### Method 3

150 g of dried industrial hemp buds were placed in a kitchen aid mixer set at 100 rpm along with 340 g water, 100 g olive oil, 6 g citric acid monohydrate and a mixture of commercial food-grade enzymes consisting of: Celluclast 1.5 L (cellulase), Ultraflow Max (betaglucanase), Peclyve (pectinase, beta-glucanases, cellulases, and beta-mannanases) and Ceremix 2XL (Alpha-amylase, Beta-glucanase, Protease). Total enzymes concentration was 3% of the hemp plant material weight. After 3.5 hours, the mixture becomes homogenous. Subsequent centrifugation of the mixture (11,000 rpm for 5 min) provided 91 g of lipid-soluble extract, 80 g of an intermediate aqueous phase and 410 g of a wet solid fraction.

The lipid-soluble extract was then washed with 0.1 N aqueous NaOH whereupon the lipid phase is discarded and the aqueous phase is then neutralized with 2 N aqueous HCl after which the crude CBDA precipitate is filtered out and centrifuged to provide a crude oleaginous CBDA of approx. 70% purity.

### Synthesis and Compound Characterization

All reactions were performed under an atmosphere of nitrogen using solvents dried by standard procedures. Reaction progress was monitored by TLC on Polygram SIL G/UV254 silica gel plates from Macherey & Nagel and on Silicagel plates 60 RP-18 F254 from Supelco. Detection of spots was effected by charring with sulfuric acid (5% in ethanol), staining by spraying the plates with an alkaline aqueous solution of potassium permanganate, staining plates in a iodine chamber or by inspection of the TLC plates under UV light (254 nm).

Preparative chromatography was performed on silica gel (0.032-0.063 mm) from Macherey & Nagel.

NMR spectra were recorded with the following spectrometers: Bruker Avance III HD 400 (1H: 400.2 MHz; 13C: 100.6 MHz), Bruker Avance III HDX 600 (1H: 600.2 MHz; 13C: 150.9 MHz) and Bruker Avance III HDX 700 (1H: 700.3 MHz; 13C: 176.1 MHz); and calibrated for the solvent signal (¹H: CDCl₃: δ=7.26 ppm; acetone-d₆: δ=2.05 ppm; dichlormethane-d₂: δ=5.32 ppm; DMSO-d₆: δ=2.50 ppm; ¹³C: CDCl₃: δ=77.16 ppm; acetone-d₆: δ=29.92 ppm; dichlormethane-d₂: δ=54.0 ppm; DMSO-d₆: δ=39.51 ppm).

ESI-TOF-HRM spectrometry was performed on a Bruker MAXIS 4G spectrometer.

Elemental analyses were obtained from a HEKAtech Euro EA 3000 apparatus.

Optical rotations were determined with a Perkin-Elmer Polarimeter 341 in a 10 cm cuvette at 20° C. with a wavelength of 589 nm (Na-lamp).

Melting points were measured with a Büchi Melting Point M-560 apparatus.

### Example 1: Isolation of CBDA-Di-Acetate from Hemp Extract

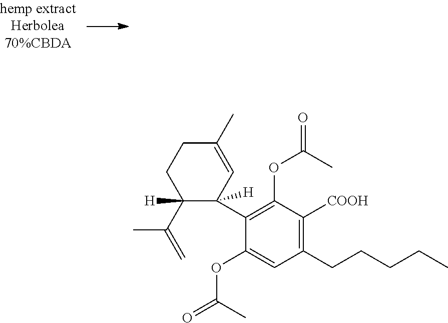 [SMILES: C=C(C)[C@@H]1CCC(C)=C[C@H]1c1c(OC(C)=O)cc(CCCCC)c(C(=O)O)c1OC(C)=O]

Five grams of Herbolea extract containing ca. 70% CBDA (w/w) was dissolved with shaking at room temperature in a mixture of DMF (10 ml) and triethylamine (10 ml) to give a deep red solution. Acetic anhydride (10 ml) was added dropwise to the stirred solution so that the temperature does not exceed 50° C. The mixture was stirred for 2 h at room temperature and cooled to 0° C. Water (10 ml) was added and the mixture was stirred for 0.5 h at room temperature. The mixture was poured into aqueous HCl solution (2 M, 50 ml) and extracted with ethyl acetate (2×35 ml). The combined ethyl acetate extracts were washed with aqueous HCl solution (2 M, 1×50 ml) followed by saturated NaCl solution (3×50 ml), dried over Na₂SO₄, filtered and concentrated. The brown oily residue (5.47 g) was dissolved in n-hexane/diethyl ether or alternatively in n-hexane/t-butyl methyl ether (1/1 v/v, 50 ml) and filtered through a layer of silica gel. The filtrate was concentrated and the oily residue was taken up with boiling (n-hexane (30 ml). After removal of heat, crystallization immediately occurs to afford CBDA-di-acetate (4.01 g, 93%) as white needles.

The melting point of CBDA-di-acetate was determined as 127° C. (literature value: 126-128° C., R. Mechoulam, Z. Ben-Zvi, Chem. Commun. 1969, 343-344).

Compound identity was confirmed by high-resolution mass spectrometry (HRMS): theoretical C₂₆H₃₃O₅[M+Na]⁺ m/z=465.22476; found: m/z=465.22486. Compound identity was confirmed by elemental analysis: calculated for C₂₆H₃₃O₅: C, 70.56, H, 7.74; found: C, 70.55, H, 7.62.

The optical rotation was measured as [a]_{D}²⁰=−68° (c=10 mg/1.5 ml; in CHCl₃).

¹H-NMR spectra and ¹³C-NMR spectra were recorded and spectral data were extracted:

¹H-NMR (CDCl₃): δ=0.80 (t, 3H, J=6.6 Hz, H-5″), 1.24, 1.25 (2 d, 4H, H-3″, 4″), 1.52 (s, 3H, COCH₃), 1.60 (s, 3H, COCH₃), 1.52-1.71 (m, 8H, H-2″, 7′, 10′), 1.96 (br.d (broad doublet), 1H, H-4a′), 2.14 (br.s (broad singlet), 3H, H-4′, 5b′), 2.61 (t, 2H, H-1″), 2.73 (br.s, 1H, H-2′), 3.39 (br.s, 1H, H-1′), 4.39, 4.49 (2 s, 2H, H-9′), 5.13 (s, 1H, H-6′), 6.77 (s, 1H, H-5), 7.19 (s, 1H, COOH). ¹³C-NMR (CDCl₃): δ=14.0 (C-5″), 20.7, 20.9 (4C, C-7′, 10′, C_{A}c), 22.4 (C-4″), 28.6 (C-3″), 30.4 (C-3′), 30.7, 31.7 (C-1″, 2″), 33.7 (C-4′), 38.7 (C-1′), 45.5 (C-2′), 111.5 (C-9′), 123.9 (C-5), 128.0 (C-5′), 133.4 (C-1), 142.0 (C-3), 147.3 (C-4), 148.5 (C-8′), 151.4 (C-2).

### Example 1: Purified CBDA

A sample of CBDA was prepared from the synthetic product according to Example 1, i.e., CBDA-di-acetate obtained after crystallization, by ester hydrolysis. The sample was analyzed by GC-MS, after derivatization of CBDA with diazomethane, and contained 99.72% CBDA with the only detectable impurity being 0.22% CBDVA.

### Example 2: Isolation of CBDA-Di-Acetate Via Carboxylation of CBD

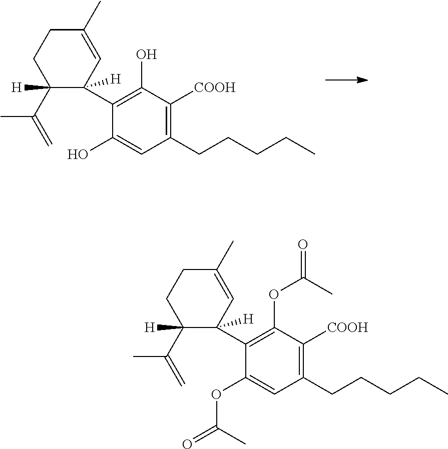

CBD (3.14 g, 10.0 mmol, purchased from American-Alb Hemp Company) was dissolved in DMF (10 ml) and magnesium methyl carbonate solution in DMF (2 M, 12.5 ml, 25.0 mmol) was added. The mixture was stirred at 130° C. for 4 h according to the procedure of R. Mechoulam, Z. Ben-Zvi, Chem. Commun. 1969, 343-344. Water (5 ml) was added, the resulting mixture was stirred for 1 h at room temperature, poured into water (100 ml) and extracted with diethyl ether (3×50 ml). The combined diethyl ether extracts were dried with Na2SO4, filtered and concentrated to afford a mixture of CBD and CBDA containing ca. 75% CBDA. The crude CBD/CBDA mixture was treated with acetic anhydride as described in Example 1 to afford CBDA-di-acetate (3.05 g, 69%).

### Example 3: Methylation of CBDA-Di-Acetate (Novel Compound)

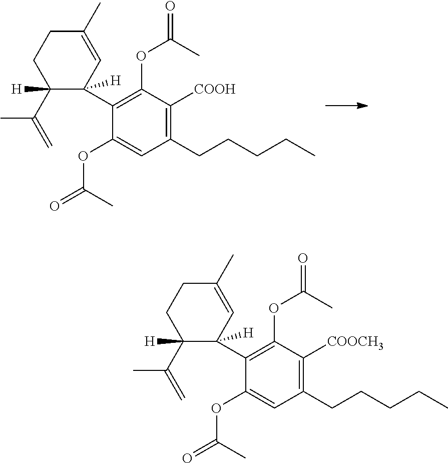

CBDA-di-acetate (10 g, 22.6 mmol) was dissolved in acetonitrile (50 ml) and triethylamine (2.52 g or 3.46 ml, 24.9 mmol, 1.1 mol-equivalents) was added with stirring at room temperature. Dimethyl sulfate (3.14 g or 2.4 ml, 24.9 mmol, 1.1 mol-equivalents) was dropped into the stirred mixture at such a rate that the temperature does not exceed 50° C., and the mixture was stirred at room temperature until TLC (RP-18 silica gel, MeOH/acetic acid 19/1) showed complete conversion of the starting material (16 h). Alternatively, the mixture can be stirred at 60° C. for 2 h. The mixture was poured into ice cold 1 M aqueous HCl solution (250 ml) and the resulting suspension was extracted with dichloromethane (2×50 ml). The combined organic extracts were successively washed with 1 M aqueous HCl solution (1×100 ml), saturated aqueous NaHCO₃ solution (2×100 ml) and brine (1×100 ml) and dried over Na₂SO₄. Filtration and concentration afforded CBDA-di-acetate-methyl-ester as a slightly yellow oil (9.8 g, 95%).

Compound identity was confirmed by high-resolution mass spectrometry (HRMS): theoretical C₂₇H₃₆O₆[M+Na]+m/z=479.24041; found: m/z=479.24105.

The optical rotation was measured as [a]_{D}²⁰=−67° (c=1.6 g/100 ml, CHCl₃).

¹H-NMR spectra and ¹³C-NMR spectra were recorded and spectral data were extracted:

¹H-NMR (CDCl₃): δ=0.91 (t, 3H, J=6.5 Hz, H-5″), 1.33 (br. s., 4H, H-3″, 4″), 1.61 (s, 3H, COCH₃), 1.70 (s, 3H, COCH₃), 1.56-1.84 (m, 2H, H-2″), 2.02-2.23 (m, 11H, H-1′, 3′, 4′, 7′, 10′), 2.61 (br. s, 1H, H-2′), 2.70 (t, 2H, J=3.9 Hz, H-1″), 3.47 (br. s, 1H, H-1′), 3.87 (s, 3H, OCH₃), 4.48, 4.58 (2 s, 2H, H-9′), 5.23 (s, 1H, H-6′), 6.84 (s, 1H, H-5). ¹³C-NMR (CDCl₃): δ=14.0 (C-5″), 20.7, 20.9 (2C, CH₃Ac), 22.4 (C-4″), 23.4 (C-7′), 28.6 (C-3″), 30.5 (2 C, C-2″, 3′), 31.7 (C-1″), 33.6 (C-4′), 38.7 (C-10′), 45.5 (C-2′), 52.2 (OCH₃), 111.5 (C-9′), 124.0 (C-5′), 133.2 (C-1), 141.0 (C-3), 147.3 (C-5), 148.0 (C-4), 150.8 (2 C, C-2, 8′), 167.1, 168.7 (C═O).

### Example 4: Deacetylation of CBDA-Di-Acetate Methyl Ester (Novel Compound)

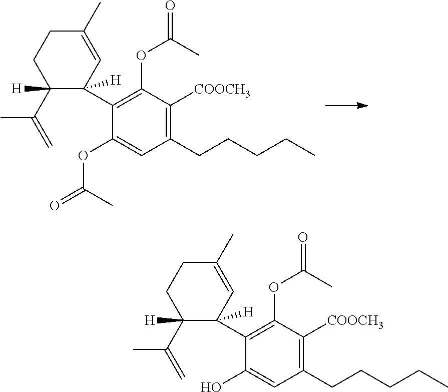

CBDA-di-acetate-methyl-ester (4.57 g, 10.0 mmol) was dissolved in methanol (20 ml), sodium methanolate (54 mg, 1.0 mmol) or a 1 M solution of ammonia in methanol (2.0 ml, 2.0 mmol ammonia) was added and the solution was stirred at room temperature for 24 h. In case of sodium methanolate, the solution was neutralized by the addition of Amberlyst IR 120 ion exchange resin (H+ form), filtered and concentrated. In case of ammonia, the solution was directly concentrated to afford the mono-deacetylated CBDA-2-acetate methyl ester (4.15 g, 100%).

Compound identity was confirmed by high-resolution mass spectrometry (HRMS): theoretical C₂₅H₃₄O₅[M+Na]+m/z=437.23039; found: m/z=437.23027.

The optical rotation was measured as [a]_{D}²⁰=−16.2° (c=0.7 g/100 ml, CHCl₃)

¹H-NMR spectra and ¹³C-NMR spectra were recorded and spectral data were extracted:

¹H-NMR (CDCl₃): δ=0.80 (t, 3H, J=6.5 Hz, H-5″), 1.23 (br. s, 4H, H-3″, 4″), 1.53 (s, 3H, COCH₃), 1.44-1.70 (m, 2H, H-2″), 1.99-2.21 (m, 11H, H-1′, 3′; 4′, 7′, 10′), 2.40-2.66 (m, 2H, H1″, 2′), 2.70, 3.47 (br. s, 1H, H-1′), 3.75 (s, 3H, OCH₃), 4.33, 4.53 (2 s, 2H, H-9′), 5.47 (s, 1H, H-6′), 6.17 (s, 1H, H-5), 6.51 (s, 1H, OH). ¹³C-NMR (CDCl₃): δ=14.0 (C-5″), 20.7 (CH_{3Ac}), 22.5 (C-4″), 23.7 (C-7′), 27.7 (C-3″), 30.8 (2 C, C-2″, 3′), 31.8 (C-1″), 33.8 (C-4′), 38.1 (C-10′), 45.6 (C-2′), 52.0 (OCH₃), 112.0 (C-9′), 122.6 (C5′), 135.2 (C-1), 141.7 (C-3), 142.6 (C-5), 146.5 (C-4), 155.9, 157.1 (C-2, 8′), 167.1, 167.4 (C═O).

### Example 5a: Cyclization of CBDA-Acetate Methyl Ester to Delta-9-THCA-Acetate Methyl Ester (Novel Compound)

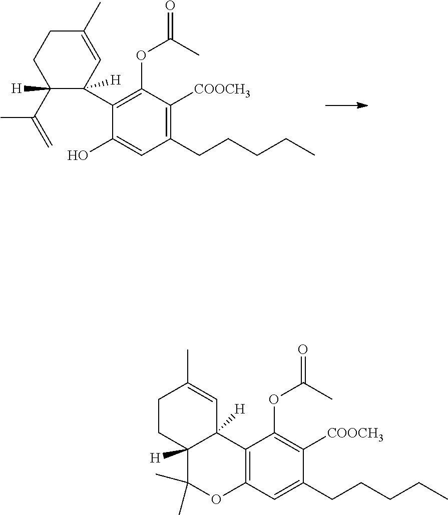 [SMILES: C=C(C)[C@@H]1CCC(C)=C[C@H]1c1c(O)cc(CCCCC)c(C(=O)OC)c1OC(C)=O ; CCCCCc1cc2c(c(OC(C)=O)c1C(=O)OC)[C@@H]1C=C(C)CC[C@H]1C(C)(C)O2]

A solution of CBDA-2-acetate methyl ester (2.07 g, 5.0 mmol), tetraethylene glycol dimethylether (0.24 ml), di-isopropylethylamine (0.24 ml) and boron trifluoride diethyl etherate (0.40 ml) in dichloromethane (50 ml) was stirred under an atmosphere of nitrogen until TLC shows complete consumption of the starting material (24 h). The solution was successively washed with saturated aqueous NaHCO₃ solution, 2 N aqueous HCl solution, saturated aqueous NaCl solution, dried over Na₂SO₄ and filtered. Concentration of the filtrate and recrystallization of the residue from n-hexane gave Delta-9-THCA-acetate methyl ester (2.0 g, 97%).

Compound identity was confirmed by high-resolution mass spectrometry (HRMS): theoretical C₂₅H₃₄O₅[M+Na]+m/z=437.22984; found: m/z=437.22970

The optical rotation was measured as [a]_{D}²⁰=−185° (c=0.9 g/100 ml, CHCl₃).

¹H-NMR spectra and ¹³C-NMR spectra were recorded and spectral data were extracted:

¹H-NMR (CDCl₃): δ=0.80 (t, 3H, J=7.0 Hz, H-5″), 1.19-1.42 (m, 4H, H-3″, 4″), 1.43-1.53 (m, 2H, H-2″), 1.53 (s, 3H, H-9′), 1.62-1.74 (m, 4H, H-6′, 10′), 1.70 (s, 3H, H-7′), 2.02 (br. s, 2H, H4′), 2.14 (s, 3H, CH₃C═O), 2.41-2.49 (m, 4H, H-1″, 5′), 2.62 (br. s, 1H, H-1′), 3.74 (s, 3H, OCH₃), 6.21 (s, 1H, H-5), 6.53 (s, 1H, H-2′).

¹³C-NMR (CDCl₃): δ=14.0 (C-5″), 19.4 (C-9′), 20.7 (CH₃Ac), 22.5 (C-4″), 23.7 (C-10′), 27.7 (C5′), 30.1 (C-4′), 30.8 (C-3″), 31.8 (C-2″), 33.8 (C-1″), 38.1 (C-1′), 45.6 (C-6′), 52.0 (OCH₃), 77.4 (C-8′), 116.0 (C-5), 117.9 (C-1), 120.5 (C-3), 122.7 (C-2′), 141.0 (C-3′), 142.6 (C-2), 146.4 (C-4), 157.1 (C-6), 167.4 (COO), 169.1 (C═O_{Ac}).

### Example 5b: Cyclization of CBDA-Acetate Methyl Ester to Delta-8-THCA-Acetate Methyl Ester (Novel Compound)

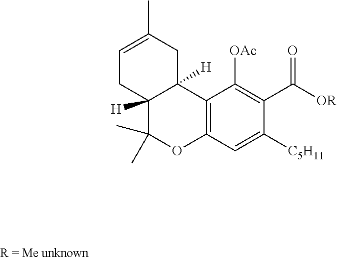

CBDA-acetate methyl ester (2.07 g, 5.0 mmol) was dissolved in toluene (10 ml), toluene-4-sulfonic acid monohydrate (10 mg, 0.05 mmol) was added and the solution heated to 80° C. with stirring for 3 h. The solution was then allowed to cool to room temperature and washed with saturated aqueous sodium hydrogen carbonate. The organic phase was then separated, dried with anhydrous sodium sulfate, filtered, and evaporated to dryness providing Delta-8 THCA-methyl ester (1.98 g, 96%).

### Example 5c: Cyclization of CBD to Delta-9-THC

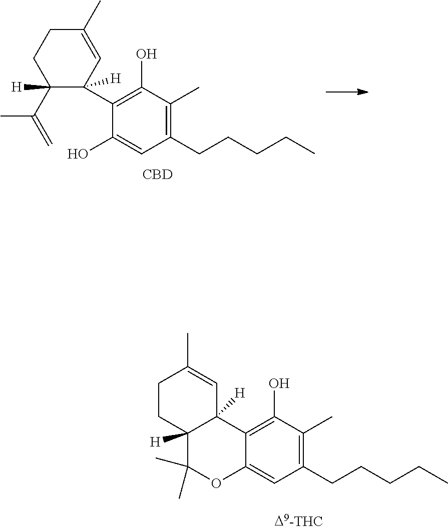

A solution of pure, crystalline CBD (10 g, 32 mmol) in dry dichloromethane (60 ml) was added at room temperature during 1 h to a vigorously stirred solution of tetraethylene glycol dimethyl ether (0.48 ml), di-isopropyl ethylamine (0.48 ml) and boron trifluoride diethyl ether complex (0.8 ml, 6.5 mmol) in dry dichloromethane (100 ml) kept under an atmosphere of nitrogen. Immediately after completion of the addition of CBD, another portion of tetraethylene glycol dimethyl ether (0.24 ml), di-isopropyl ethylamine (0.24 ml) and boron trifluoride diethylether complex (0.4 ml, 3.3 mmol) was added and the mixture was stirred for an additional 3 h under nitrogen until thin-layer chromatography (TLC) (RP-C₁₈, methanol/acetic acid 19/1 vol/vol) indicated complete conversion of the starting material. The mixture was first washed with saturated aqueous NaHCO₃ solution (1×80 ml), next with 5% aqueous HCl solution (1×80 ml) and finally three times with saturated NaCl solution (3×200 ml). Repeated washing with saturated NaCl solution was necessary for completely removing the reagent combination of tetraethylene glycol dimethylether and di-isopropyl ethylamine. The organic phase was dried over Na₂SO₄, filtered and concentrated in vacuo to afford Delta-9-Tetrahydrocannabinol (9.89 g, 99% yield) as a clear, slightly yellowish oil.

Compound identity was confirmed by HRMS: theoretical C₂₁H₃₀O₂[M+Na]+m/z=314.2246; found: m/z=314.2247.

The optical rotation was measured as [a]_{D}²⁰=−151° (c=1.0 g/100 ml, CHCl₃).

Compound purity was confirmed by GC-MS: >99% Delta-9-THC; Delta-8-iso-THC and Delta-8-THC were not detectable.

### Example 5d: Cyclization of CBD to Delta-8-THC Using SiOx-BA

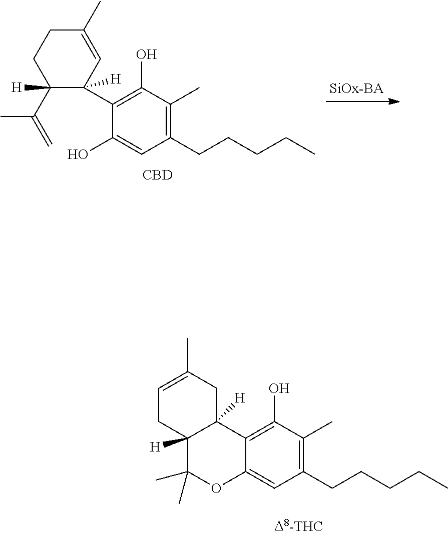

### Reagent SiOx-BA

A solution of boron trifluoride diethyl etherate (4 ml, 3.48 g, 24.5 mmol) in 35 ml DCM was added to oven-dried silica gel (10 g) and the mixture was dried in vacuo at a rotary evaporator until the silica gel became lively and behaved like a liquid. Next, the silica gel was mixed with a solution of p-toluene sulfonic acid mono hydrate (8.0 g, 42 mmol) in ethanol (16 ml). This mixture is again dried in vacuo at a rotary evaporator with the bath temperature set to 60° C. to yield the reagent “SiOx-BA”.

### Delta-8-Tetrahydrocannabinol

A solution of CBD (10.0 g, 31.8 mmol) in dichloromethane (40 ml) was shaken with SiOx-BA (10 g) at room temperature until TLC (RP C₁₃, MeOH/AcOH 19/1 vol/vol) indicated complete conversion of the starting material. The mixture was filtered, and the filtrate was washed with aqueous NaHCO₃ solution, dried over Na₂SO₄ and filtered. Concentration of the filtrate afforded Delta-8-Tetrahydrocannabinol (9.8 g, yield 98%).

### Analysis

The optical rotation was measured as [a]_{D}²⁰=−283° (c=1.0 g/100 ml, CHCl₃). This value is identical to the one reported in the literature (Marzullo, P.; Foschi, F.; Coppini, D. A.; Fanchini, F.; Magnani, L.; Rusconi, S.; Luzzani, M.; Passarella, D., J. Nat. Prod. 83, 2020, 2894-2901).

NMR data were identical to those reported in the literature (Marzullo, P.; Foschi, F.; Coppini, D. A.; Fanchini, F.; Magnani, L.; Rusconi, S.; Luzzani, M.; Passarella, D., J. Nat. Prod. 83, 2020, 2894-2901).

Compound identity was further confirmed by electron ionization mass spectrometry (EI-MS): m/z=314 [M]⁺, 299 [M−CH₃]⁺, 271 [M−C₃H₇]⁺, 258 [M−C₄H₈]⁺, 246 [M−C₅H₈]+(using a retro Diels-Alder reaction characteristic for Delta-8-THC), 231 [M−C₆H₁₁]+.

### Example 6: Conversion of the THCA-Acetate Methyl Ester into THCA-Carbonate (Novel Compound)

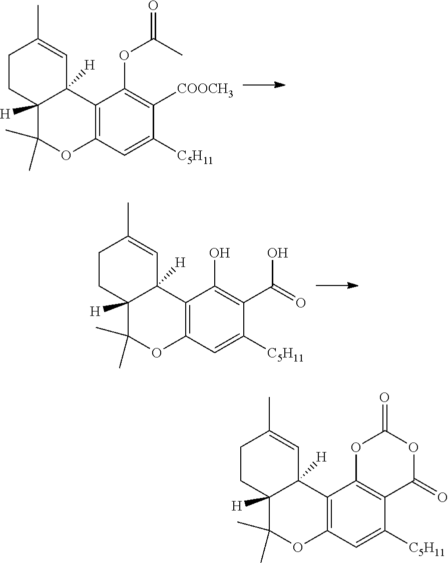

THC-acetate methyl ester (1.0 g, 2.4 mmol) was stirred for 24 h in a 1 M solution of LiOH in methanol (20 ml). The resulting solution was poured into 2 M aqueous HCl solution (100 ml) and extracted with dichloromethane (2×50 ml). The combined extracts were washed with saturated aqueous NaCl solution, dried over Na2SO4, filtered and concentrated to afford crude THCA. The crude THCA was dissolved in toluene (20 ml), trimethylamine (0.4 g, 4.0 mmol) and 2 M phosgene in toluene (2 ml, 4 mmol) was added. The mixture was stirred at room temperature until all THC was consumed, washed with saturated aqueous NaHCO₃ solution and dried over Na2SO4. Filtration and concentration of the filtrate afforded THCA carbonate (0.77 g, 83%).

Compound identity was confirmed by high-resolution mass spectrometry (HRMS): theoretical C₂₃H₂₈O₅[M+Na]+m/z=407.18344; found: m/z=407.18350.

The optical rotation was measured as [a]_{D}²⁰=−50.1° (c=1 g/100 ml; in CHCl₃).

¹H-NMR spectra and ¹³C-NMR spectra were recorded and spectral data were extracted. Significant NMR signals are provided. The obtained THCA carbonate displays insufficient solubility in CDCl₃ and decomposition in d6-DMSO.

¹H-NMR (CDCl₃) significant peaks: δ=0.91 (t, 3H, J=7.0 Hz, H-5″), 1.20-1.45 (m, 4H, H-3″, 4″), 1.55 (s, 3H, H-9′), 1.65 (s, 3H, H-7′), 5.37 (s, 1H, H-2′). ¹³C-NMR (CDCl₃) significant signals: δ=14.1 (C-5″), 22.3 (C-7′), 25.1 (C-9′), 124.0 (C-2′), 135.4 (C-3′), 146.5, 149.4 (C═O).

### Example 7: Synthesis of CBDA from CBD

100 mmol CBD (31.5 g) was dissolved in 100 ml acetonitrile and 300 mmol of DBU (1,8-Diazabicyclo[5.4.0]undec-7-ene) (45.6 g) was added. The solution was then gassed with carbon dioxide bubbled through the solution. This produces CBDA in greater than 99% purity and yield. The work-up involved washing with 5 wt. % aqueous hydrogen chloride followed by brine, extracting into an organic solvent, drying the extract with anhydrous sodium sulphate and evaporating the solvent to produce CBDA in greater than 99% purity. The sample purity was confirmed by GC-MS after derivatization of CBDA with diazomethane.

The identity of CBDA was confirmed by ¹H-NMR (CDCl₃) NMR and ¹³C-NMR (CDCl₃) in agreement with data published by Formato et al. (Molecules 25, 2020, 2638; FIG. 6).

### Compounds and Abbreviations

CBD; Cannabidiol; 2-[(1R,6R)-3-methyl-6-prop-1-en-2-ylcyclohex-2-en-1-yl]-5-pentylbenzene-1,3-diol.

CBDA; Cannabidiolic acid; 2,4-dihydroxy-3-[(1R,6R)-3-methyl-6-prop-1-en-2-ylcyclohex-2-en-1-yl]-6-pentylbenzoic acid.

THC; delta-9 Tetrahydrocannabinol; (6aR,10aR)-6,6,9-trimethyl-3-pentyl-6a,7,8,10a-tetrahydrobenzo[c]chromen-1-ol.

THCA; delta(9)-Tetrahydrocannabinolic acid; (6aR,10aR)-1-hydroxy-6,6,9-trimethyl-3-pentyl-6a,7,8,10a-tetrahydrobenzo[c]chromene-2-carboxylic acid.

THCV; Tetrahydrocannabivarin; (6aR,10aR)-6,6,9-trimethyl-3-propyl-6a,7,8,10a-tetrahydrobenzo[c]chromen-1-ol.

THCVA; Tetrahydrocannabivarinic acid; (6aR,10aR)-1-hydroxy-6,6,9-trimethyl-3-propyl-6a,7,8,10a-tetrahydrobenzo[c]chromen-2-carboxylic acid.

THCP; Tetrahydrocannabiphorol; (6aR,10aR)-6,6,9-trimethyl-3-heptyl-6a,7,8,10a-tetrahydrobenzo[c]chromen-1-ol.

THCPA; delta(9)-Tetrahydrocannabiphorolic acid; (6aR,10aR)-1-hydroxy-6,6,9-trimethyl-3-heptyl-6a,7,8,10a-tetrahydrobenzo[c]chromene-2-carboxylic acid.

1. A compound according to Formula I 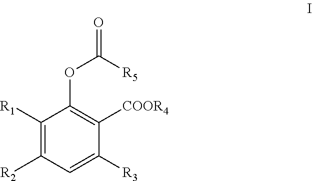
wherein R¹=methyl-4-(prop-1-en-2-yl)-cyclohex-1-ene-3-yl (limonenyl);
wherein R²=acetate, propionate, butyrate, or OH;
wherein R³=propyl, pentyl, or heptyl;
wherein R⁴=methyl, ethyl, or propyl;
wherein R⁵=methyl, ethyl, or propyl,
or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, ethyl, or propyl, and wherein R⁵=methyl, ethyl, or propyl,
or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ are radicals forming a direct bond to each other.

2. The compound according to claim 1, having Formula I 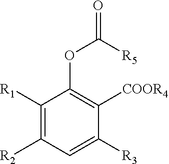
wherein R¹=methyl-4-(prop-1-en-2-yl)-cyclohex-1-ene-3-yl (limonenyl);
wherein R²=acetate, or OH;
wherein R³=propyl, pentyl, or heptyl;
wherein R⁴=methyl;
wherein R⁵=methyl,
or
wherein R¹=methyl-4-(2-propyl)-cyclohex-1-ene-3-yl or methyl-4-(2-propyl)-cyclohex-5-ene-3-yl, wherein R²═O, and R¹ and R² together form a ring structure in which R² is an internal ring atom, wherein R³=propyl, pentyl, or heptyl, wherein R⁴=methyl, and wherein R⁵=methyl,
or
wherein R¹=methyl-4-(2-propyl)-cyclohex-1-ene-3-yl, wherein R²═O, and R¹ and R² together form a ring structure in which R² is an internal ring atom, wherein R³=propyl, pentyl, or heptyl, wherein R⁴ and R⁵ are radicals forming a direct bond to each other.

3. The compound according to claim 1, selected from

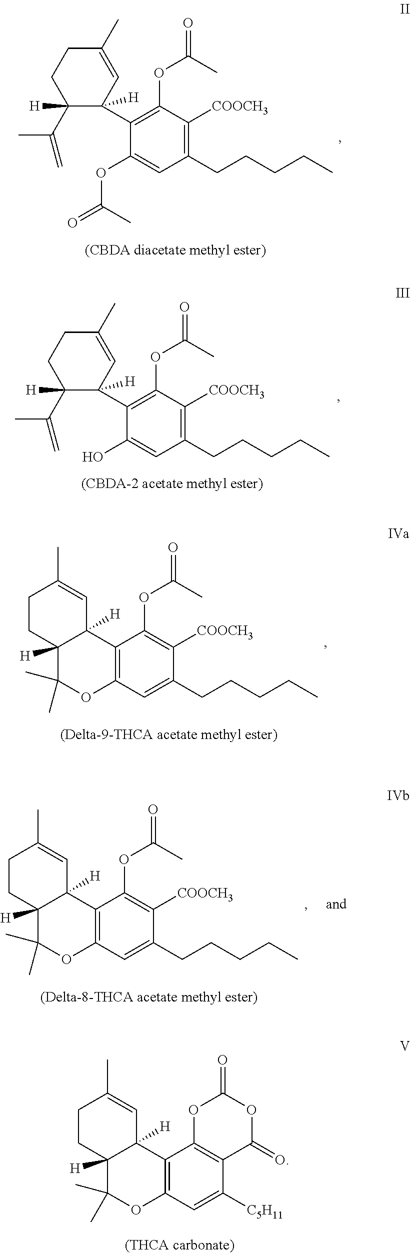

4. A process for the synthesis and/or purification of tetrahydrocannabinolic acid (THCA) alkanoate and/or cannabidiolic acid (CBDA) di-alkanoate, tetrahydrocannabinolic acid (THCA) acetate and/or cannabidiolic acid (CBDA) diacetate, comprising the steps of:
providing a crude plant extract containing a mixture of cannabinoic acids, or providing CBDA;
contacting and/or reacting the crude plant extract or the CBDA with an acylation agent and a chemical base, to obtain THCA alkanoate and/or CBDA di-alkanoate; and
isolating the obtained THCA alkanoate and/or CBDA di-alkanoate,
wherein the acylation agent is selected from the list consisting of C₁-C₁₀ aliphatic and aromatic anhydrides,
wherein the chemical base is a tertiary amine,
wherein isolation is carried out by means of crystallization.

5. The process according to claim 4, wherein crystallization is performed in n-pentane or n-hexane.

6. The process according to claim 4, wherein the crude plant extract is provided by enzymatic extraction.

7. The process according to claim 4, further comprising treating the obtained CBDA di-acetate with an alkylation agent to obtain a first CBDA diacetate derivative.

8. The process according to claim 7, further comprising deacylating the first CBDA di-acetate derivative to obtain a CBDA mono-acetate derivative.

9. The process according to claim 8, further comprising converting the CBDA mono-acetate derivative into a THCA mono-acetate derivative.

10. The process according to claim 9, further comprising converting the THCA mono-acetate derivative into THCA carbonate according to Formula V

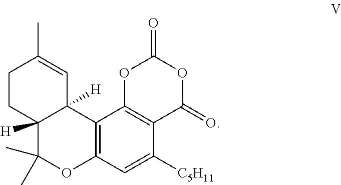

11. The process according to claim 7, wherein the obtained derivatives are at least 70% pure.

12. The process according to claim 4, wherein a distillation step is absent and/or wherein a chromatography step is absent.

13. The process according to claim 4, wherein the crude plant extract is obtained from a plant of the species Cannabinaceae.

14. (canceled)

15. A method of treating or preventing a disease associated with a cannabinoid receptor, the method comprising administering a composition comprising the compound of claim 1 to a subject in need thereof, wherein the cannabinoid receptor is one or more of CB1, CB2, 5HT1A, 5HT2A, GPR18, GPR55, GPRI 19, TRPV1, TPRV2, PPARγ or a μ-opioid receptor.

16. The method according to claim 15, wherein the subject suffers from a condition relating to or benefiting from COX-2 inhibition, wherein the compound is CBDA-2-acetate.

17. The process according to claim 4, wherein the contacting and/or reacting the crude plant extract with the acylation agent and the chemical base comprises dissolving the crude plant extract by shaking at room temperature in a suitable solvent, adding the acylation agent dropwise under stirring and maintaining the solvent temperature below 50° C., and allowing further contact and/or reaction for 1 to 4 hours at room temperature.

18. A process for the conversion of CBD into Delta-9-THC comprising the steps of:
providing a first solution comprising CBD in an organic solvent;
providing a second solution comprising tetraethylene glycol dimethyl ether, diisopropyl ethylamine and a boron trifluoride diethyl ether complex dissolved in the organic solvent;
adding the first solution to the second solution to obtain a reaction mixture;
adding a further portion of the second solution to the reaction mixture; and
obtaining Delta-9-THC,
wherein the process is carried out at a temperature between 1° and 40° C., wherein the process is carried out under a protection gas atmosphere.

19. A process for the synthesis of cannabidiolic acid (CBDA) comprising the steps of:
providing a solution comprising CBD in an organic solvent;
adding a non-nucleophilic base to the solution, wherein the non-nucleophilic base is DBU (1,8-Diazabicyclo[5.4.0]undec-7-ene); and
gassing carbon dioxide, through the solution to obtain CBDA.

20. The process according to claim 19, comprising the further steps of:
washing the solution with a neutralizing solution;
repeated washing of the solution with a NaCl solution; and
extracting the solution into a second organic solvent to obtain an extract, and evaporating the second organic solvent from the extract to obtain CBDA.
